(19) United States
(12) Patent Application Publication
(10) Pub. No.: US 2005/0267447 A1
(43) Pub. Date: Dec. 1, 2005

(54) Harrison improved female self-catheter

(76) Inventor: Nora T. Harrison, Honolulu, HI (US)

Correspondence Address: Nora T. Harrison Ed. D., 532B Kawaihae St., Honolulu, HI, 96825, US

(21) Appl. No.: 11/113,421

(22) Filed: Apr. 25, 2005

(51) Int. Cl. A61M 27/00

(52) U.S. Cl. 604/544

(57) ABSTRACT

The Harrison Improved Female Self.catheter has the improvement of an application of an approximately one-inch wide band of an anti.infection medicinal coating, ex. liquid Cipro, applied around the external wall of an approximately six-inch long female self.catheter, starting approximately 1½ inches from the insertable tip of the female self.catheter to minimize or to eliminate any infection or source of infection when said treated self.catheter is inserted into a retentive or non-retentive urinary bladder when used to remove urine from a retentive or a non-retentive urinary bladder when used under the instructions of a physician. This invention cannot be supported by any other claim.

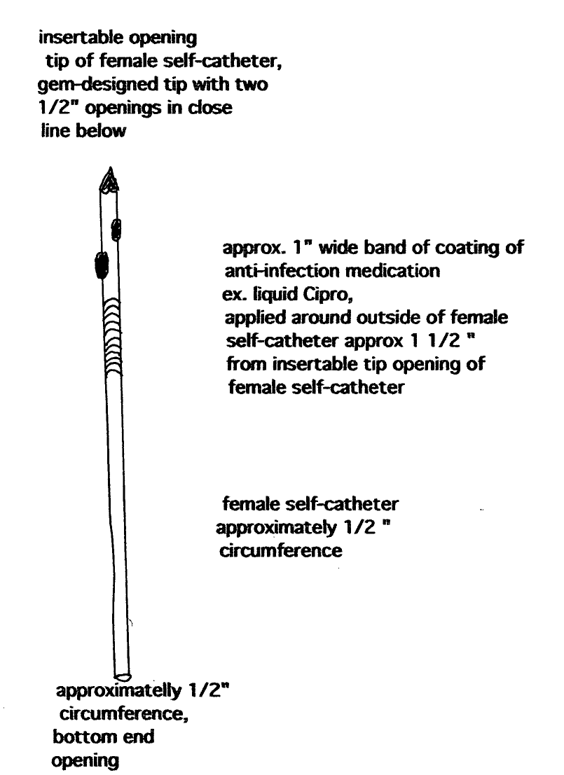

What I claim as my invention is the improved model of a 6″ long female self-catheter which is improved by applying an approximately one-inch wide band of coating or application of an anti-infection medication ex. liquid Cipro, around the outside of the female self-catheter, starting approximately 1½inches from the insertable tip of the female self-catheter. Claim #1.

When the Harrison Improved Female Self-catheter is inserted into the vagina, the resulting release of said-medication, ex. liquid Cipro, will minimize or indeed could eliminate, a possible source of infection and/or the infection itself, if an infection is already present in the vagina, when the Harrison Improved Female Self-Catheter is used according to a physician's instructions. Claim #2.

The inventor, herself, has direct knowledge of the use of a female self-catheter under the instruction of a physician in treating a retentive bladder. Claim #3.

1. What I claim to be my invention is the improved model of a 6 inch long female self.catheter which is improved by applying an approximately One-inch wide band of coating or application of an anti.infection Medication ex liquid Cipro around the (outside) external wall of the Female self.catheter, starting approximately 1½inches from the insertable tip of the female self.catheter.
When the Harrison Improved Female Self.Catheter is inserted into the (vagina) urinary bladder, the resulting release of said medication, ex liquid Cipro, will minimize or indeed could eliminate a possible source, and, or, the infection itself, if an infection is already present in the (vagina) urinary bladder or tract when the Harrison Improved Female Self.catheter is used according to a physician's instructions.
The inventor, herself, has direct knowledge of the use of a female self. Catheter under the instruction of a physician in treating a (retentive Bladder) retentive urinary bladder.
This invention cannot be supported by an other claim.
